(19) United States
(12) Patent Application Publication
(10) Pub. No.: US 2008/0138393 A1
(43) Pub. Date: Jun. 12, 2008

(54) Water soluble extract of spinach for prevention and repair of DNA damage

(75) Inventors: Jesse C. Leverett, Rockford, MI (US); Rodney M. Johnson, Temecula, CA (US); Stephen R. Missler, Grand Rapids, MI (US); David J. Fast, Grand Rapids, MI (US); Tom La, Murrieta, CA (US); John V. Scimeca, Kentwood, MI (US)

Correspondence Address: IN RE: ALTICOR INC. 28533;BRINKS, HOFER, GILSON & LIONE, P.O. BOX 10395, CHICAGO, IL, 60610, US

(73) Assignee: Access to Business Group International LLC

(21) Appl. No.: 11/698,016

(22) Filed: Jan. 25, 2007

Related U.S. Application Data
(63) Continuation-in-part of application No. 11/636,889, filed on Dec. 11, 2006.

(51) Int. Cl. A61K 36/00; A61K 9/127; A61K 36/31; A61K 38/47; A61P 43/00

(52) U.S. Cl. 424/450; 424/725; 424/755; 424/774

(57) ABSTRACT

Compositions comprising a water soluble spinach extract, compositions comprising a water soluble spinach extract, a liposome, a cardiolipin, and at least one antioxidant, compositions comprising a water soluble spinach extract, an extract of Arabidopsis thaliana or of the mustard (Brassica) plant, a liposome, a cardiolipin, and at least one antioxidant, processes for obtaining such compositions, and methods of using such compositions to repair damage to nuclear DNA, mitochondrial DNA, or both, prevent or decrease damage to such DNA from, for example, reactive oxygen species or 8-hydroxydeoxyguanosine and/or to repair or prevent mitochondrial damage and loss of membrane fluidity are disclosed. Compositions of the present invention may be topically administered, orally administered or parenterally, such as administration by injection. When topically administered, additives such as penetration enhancers, fragrances, preservatives, moisturizers and cosmetic adjuvants may be included in compositions of the present invention.

This application is a continuation-in-part and claims priority to U.S. patent application Ser. No. 11/636,889, filed Dec. 11, 2006, the entire contents of which are hereby incorporated by reference.

### BACKGROUND

Mitochondria are the site of energy production within the cell. They are also the site of extreme free radical activity from the by-products of oxidative respiration, reactive oxygen species (ROS). Free radical activity in both cells and mitochondria can lead to cellular and/or mitochondrial damage with resultant loss of energy product, DNA malfunction, or up-regulation of destructive enzyme pathways. The result is accelerated aging and loss of vital cellular functionality.

One of the most important markers for ROS-mediated DNA damage is 8-hydroxydeoxyguanosine (8-OHdG). Specifically, oxidizing agents can convert guanine to 8-OHdG which introduces mutations in the DNA by allowing guanine to now pair with adenosine instead of cytosine. Thus, when DNA is replicated the guanosine-cytosine pairing in the sequence is replaced by an adenosine-thymine pairing. Thus, 8-OHdG is a known cause of base mispairing, random point mutations and nucleic acid deletions.

Since DNA encodes vital cellular peptides, unchecked DNA damage can accumulate in a cell and eventually will lead to cellular malfunction or death. Fortunately, cells and mitochondria have repair mechanisms to deal with DNA damage caused by, for example, 8-OHdG and reactive oxygen species. However, the repair mechanisms are not perfect, especially in the mitochondria itself. It has been reported that even with normal repair mechanisms in place, there is about a 10 fold greater level of mitochondrial DNA (“mtDNA”) damage compared to nuclear DNA damage. The mechanisms of DNA repair in mitochondria have gained a great deal of interest in recent years due to the recognition that some aspects of aging and certain diseases inherited as mitochondrial DNA mutations are exacerbated by accumulations of errors in mitochondrial sequences over the life of the individual.

There currently are two primary ways to address concerns over DNA damage to the cells. The first is to bolster levels of antioxidants in order to scavenge destructive molecules before they damage the DNA or other subcellular structures. The second is to either add directly or cause up-regulation of the family of DNA repair enzymes responsible for excision and repair of damaged DNA strands. Some examples of these types of treatments are: LS DNage from Laboratories Serobiologiques and the repair enzymes available from AGI Dermatics. LS DNage from Laboratories Serobiologiques is reported to up-regulate gadd-45 (growth arrest and DNA damage) formation. Gadd-45 is a multifunctional protein that has been shown to modulate the activity and accessibility of certain DNA repair enzymes. AGI Dermatics offers three classes of repair enzymes. The first is an endonuclease responsible for excision of thymine dimers. The second is a photoreactivating enzyme which acts to directly reverse the thymine dimer formations. The third is ogg-1 (8-oxoguanine DNA glycosylase) isolated from Arabidopsis thaliana, which targets excision repair of 8-OHdG. Although these approaches are somewhat effective at repairing specific types of DNA damage, they are incomplete. Specifically, these solutions either allow the cell to become oxidatively stressed and then work to repair the damage, or they seek to prevent oxidative stress but fail to repair the damage once it occurs.

### BRIEF SUMMARY

In one example, the present invention is a composition comprising a water soluble spinach extract, wherein the water soluble spinach extract prevents or decreases oxidative damage of nuclear DNA, mitochondrial DNA, or both; repairs damage of nuclear DNA, mitochondrial DNA, or both; and/or promotes or increases production or synthesis of ATP.

In another example, the present invention is a method of preventing or decreasing oxidative damage of nuclear DNA, mitochondrial DNA, or both comprising administering a composition comprising a water soluble spinach extract, wherein the water soluble spinach extract prevents or decreases oxidative damage of nuclear DNA, mitochondrial DNA, or both.

In a further example, the present invention is a method of repairing oxidative damage to nuclear DNA, mitochondrial DNA, or both, comprising administering a composition comprising a water soluble spinach extract, wherein the water soluble spinach extract repairs oxidative damage to nuclear DNA, mitochondrial DNA, or both.

In another example, the present invention is a method of increasing ATP production or synthesis in a cell comprising administering a composition comprising a water soluble spinach extract, wherein the water soluble spinach extract increases ATP production or synthesis in the cell.

In a further example, the present invention is a composition comprising a water soluble spinach extract, wherein the water soluble spinach extract is obtained by microfiltration of spinach.

In yet a further example, the present invention is a composition comprising a water soluble spinach extract, wherein the water soluble spinach extract is obtained by milling fresh, frozen, dehydrated, or dried spinach and water to form a slurry, separating the solid and liquid components of the slurry, collecting the liquid component of the slurry, placing the liquid component of the slurry in a steam kettle, adding active carbon (i.e. activated carbon, activate carbon) to the steam kettle, mixing the liquid component of the slurry with the active carbon in the steam kettle using steam and possibly an agitator, and separating the liquid component from the active charcoal and microfiltering, for example nanofiltering, the liquid component to obtain a water soluble spinach extract, and further wherein the water soluble spinach extract prevents or decreases oxidative damage of nuclear DNA, mitochondrial DNA, or both, repairs damage of nuclear DNA, mitochondrial DNA, or both, and/or promotes or increases production or synthesis of ATP.

In a still further example, the present invention is a composition comprising a water soluble spinach extract obtained using the process described in the preceding paragraph, wherein the process further includes a pasteurization step and/or a step of adding a carrier, for example, butylene glycol (1,3-Butanediol; 1,3-Butylene glycol) and/or preservatives. Some examples of preservatives that may be used in compositions of the present invention include a 2-phenoxyethanol such as Phenonip® (Clariant Corp. Charlotte, N.C.), and chlorphenesin, for example, Germazide® M (Engelhard Corp., Iselin, N.J.).

In another example, the present invention is a composition comprising a water soluble spinach extract, a liposome, a cardiolipin, and at least one antioxidant, wherein the cardiolipin is embedded in a phospholipid bilayer of the liposome, and further wherein the at least one antioxidant is embedded in a phospholipid bilayer of the liposome along with the cardiolipin, is contained in an aqueous center of the liposome, or both. In one example the cardiolipin may be tetraoleoyl-cardiolipin, tetrapalmitoleoyl-cardiolipin, tetramyristoyl-cardiolipin, or seed oil derived cardiolipin; the liposome may be primarily composed of phosphatidyl choline; the at least one antioxidant embedded in the phospholipid bilayer along with the cardiolipin may be methylgentisate (or methyl gentisate or methyl dehydroxybenzoate); and the at least one antioxidant contained in the aqueous center of the liposome may be I-carnosine.

In a further example, the present invention is a method of improving, maintaining or restoring mitochondrial function and repairing oxidative damage to DNA comprising administering a composition comprising a water soluble spinach extract, a liposome, a cardiolipin, and at least one antioxidant, wherein the cardiolipin is embedded in a phospholipid bilayer of the liposome and the at least one antioxidant is embedded in a phospholipid bilayer of the liposome, contained in an aqueous center of the liposome, or both. In a further example the cardiolipin may be tetraoleoyl-card iolipin, tetrapalmitoleoyl-card iolipin, tetramyristoyl-card iolipin, or seed oil derived cardiolipin; the liposome may be primarily composed of phosphatidyl choline; the at least one antioxidant embedded in the phospholipid bilayer along with the cardiolipin may be methylgentisate (or methyl gentisate or methyl dehydroxybenzoate); and the at least one antioxidant contained in the aqueous center of the liposome may be I-carnosine.

In another example, the present invention is a composition comprising a water soluble spinach extract, an extract of Arabidopsis thaliana, for example 8-oxoguanine DNA glycosylase (a DNA repair enzyme that targets excision repair of 8-OHdG), a liposome, a cardiolipin, and at least one antioxidant, wherein the cardiolipin is embedded in a phospholipid bilayer of the liposome, and further wherein the at least one antioxidant is embedded in a phospholipid bilayer of the liposome along with the cardiolipin, is contained in an aqueous center of the liposome, or both. In one example the cardiolipin may be tetraoleoyl-cardiolipin, tetrapalmitoleoyl-cardiolipin, tetramyristoyl-cardiolipin, or seed oil derived cardiolipin; the liposome may be primarily composed of phosphatidyl choline; the at least one antioxidant embedded in the phospholipid bilayer along with the cardiolipin may be methylgentisate (or methyl gentisate or methyl dehydroxybenzoate); and the at least one antioxidant contained in the aqueous center of the liposome may be I-carnosine. Another example of the present invention is a method of using the composition described in this paragraph, for example, to improve, maintain or restore mitochondrial function and/or repair oxidative damage to cellular and/or mitochondrial DNA.

In a further example, the present invention is a composition comprising a water soluble spinach extract, an extract of the mustard (Brassica) plant, a liposome, a cardiolipin, and at least one antioxidant, wherein the cardiolipin is embedded in a phospholipid bilayer of the liposome, and further wherein the at least one antioxidant is embedded in a phospholipid bilayer of the liposome along with the cardiolipin, is contained in an aqueous center of the liposome, or both. In one example the cardiolipin may be tetraoleoyl-cardiolipin, tetrapalmitoleoyl-cardiolipin, tetramyristoyl-cardiolipin, or seed oil derived cardiolipin; the liposome may be primarily composed of phosphatidyl choline; the at least one antioxidant embedded in the phospholipid bilayer along with the cardiolipin may be methylgentisate (or methyl gentisate or methyl dehydroxybenzoate); and the at least one antioxidant contained in the aqueous center of the liposome may be I-carnosine. Another example of the present invention is a method of using the composition described in this paragraph, for example, to improve, maintain or restore mitochondrial function and/or repair oxidative damage to cellular and/or mitochondrial DNA.

### BRIEF DESCRIPTION OF THE DRAWINGS

FIG. 1 is a flow chart illustrating a process that may be used to obtain a water soluble extract used in compositions of the present invention.

FIG. 2 is a flow chart illustrating a variation on the process that may be used to obtain a water soluble extract and frozen spinach is a starting material.

FIG. 3 is a flow chart illustrating another variation on the process that may be used to obtain a water soluble extract and spinach dehydrate is a starting material.

FIG. 4 is a graph illustrating ATP production achieved using a water soluble extract of spinach dehydrate comprising approximately 50% spinach extract, approximately 49% butylene glycol, and approximately 1% preservative, for example Phenonip® or Germazide® M. The results show that the water soluble spinach dehydrate extract induces a dose dependent increase in cellular ATP levels compared to untreated control cells. In FIG. 4, data are expressed as % control ATP compared to the ATP levels in untreated control cells. An increase in ATP is therefore considered a positive effect of the sample(s) tested.

FIG. 5 is a bar graph illustrating ATP production achieved using various water soluble spinach extracts. The results show that water soluble spinach extracts induce a dose dependent increase in cellular ATP levels compared to untreated control cells. In FIG. 5, data are expressed as % control ATP compared to the ATP levels in untreated control cells. An increase in ATP is therefore considered a positive effect of the sample(s) tested.

### DETAILED DESCRIPTION

It is to be understood that this invention is not limited to the particular compositions, methodology, or protocols described herein. Further, unless defined otherwise, all technical and scientific terms used herein have the same meaning as commonly understood to one of ordinary skill in the art to which this invention belongs. It is also to be understood that the terminology used herein is for the purpose of describing particular examples only, and is not intended to limit the scope of the present invention, which will be limited only by the claims.

The present invention overcomes the disadvantages of existing technologies for addressing nuclear and/or mitochondrial DNA repair by providing a more comprehensive approach to preserving cellular function by protecting the cell from oxidative stress and by working to repair oxidatively damaged DNA, for example DNA damaged by 8-OHdG. Specifically, the present invention seeks to improve cellular function, particularly mitochondrial function, by preventing damage to the DNA, both nuclear and mitochondrial, through bolstering the cell's resistance to oxidative stress and repair of oxidative damage to DNA. More specifically, the present invention is a composition comprising a unique water soluble extract of spinach, wherein the water soluble spinach extract facilitates repair of oxidative DNA damage, specifically 8-OHdG damage. In one example, the present invention is a complex that targets mitochondria (a “mitocomplex” or “mitocom”) and/or is designed to fortify cellular membranes and structures (i.e. cellufortification). Such a complex may be comprised of a water soluble spinach extract, an extract of mustard (Brassica) seed or an extract of Arabidopsis thaliana, for example Roxisome® (AGI Dermatics, Inc., Freeport, N.Y.) or 8-oxoguanine DNA glycosylase (a DNA repair enzyme that targets excision repair of 8-OHdG), and a liposome, composed primarily of phosphatidylcholine, having cardiolipin and methyl dehydroxybenzoate (methylgentisate) embedded in the phospholipid bilayer of the liposome and I-carnosine embedded in the aqueous center of the liposome.

A “water soluble extract” is any extract of spinach that is soluble or capable of dissolving in water regardless of the method used to obtain the extract. Thus, water soluble extracts of spinach may be obtained, for example, by a steam distillation process, a nanofiltration process, a cold water extraction process, a hot water extraction process, extraction with organic solvents etc. One of ordinary skill in the art will appreciate that there are many other methods or extraction processes that might be used to obtain a water soluble extract within the scope of the present invention.

Spinach (Spinacia oleracea) is generally considered one of the most important antioxidative vegetables. It is estimated that freshly cut spinach leaves contain approximately 1000 mg of total flavonoids per kilogram. Many different flavonoids are present in spinach including, for example, patuletin (3,5,7,3′,4′-pentahydroxy-6-methoxyflavone), spinacetin, flavonol glycosides, glucuronides, acylated di-and triglycosides of methylated and methylene dioxide derivatives of 6-oxygenated flavonols. In addition to such flavonoids, a powerful, water soluble, natural antioxidant mixture, known as “NAO”, is found in spinach. NAO specifically inhibits the lipoxygenase enzyme and the antioxidative activity of NAO has been compared to that of other known antioxidants and found to be superior in vitro and in vivo to that of green tea, N-acetylcysteine, butylated hydroxytoluene and vitamin E. Lomnitski et al., “Composition, Efficacy, and Safety of Spinach Extracts.” Nutrition and Cancer; 2003; 46(2):222-231.

The antioxidative properties of spinach are important because oxidative damage to cells and DNA increases with age and is considered to be a significant contributor to the aging process, and several diseases, including cancer. Oxidative damage to DNA can be caused by excited oxygen species, which are produced by radiation or are by-products of aerobic metabolism. The oxidized base 8-OHdG, one of approximately 20 known radiation damage products, has been found to be more prevalent in mitochondrial DNA than nuclear DNA, though it is found in both types of DNA. Richter et al., “Normal oxidative damage to mitochondrial and nuclear DNA is extensive.” Proc. Natl. Acad. Sci. USA. 1988;85:646-6467. The high levels of 8-OHdG in mt DNA may be caused by the immense oxygen metabolism occurring in mitochondria, relatively inefficient repair of mtDNA, and likely is responsible for the high mutation rate observed for mtDNA. Thus, there is a need for compositions that prevent mutations caused by 8-OHdG and/or for compositions that repair DNA, for example mitochondrial DNA, damaged by 8-OHdG.

As explained above, oxidizing agents, such as ROS, can convert guanine residues in DNA to 8-OHdG. The new 8-OHdG residue in the DNA introduces mutations in the DNA by causing a base pairing modification, specifically by pairing with adenosine instead of cytosine (which normally pairs with guanine). Thus, when DNA is replicated the guanine-cytosine pairing in the sequence is replaced by an adenosine-thymine pairing. Significantly, the water soluble spinach extract of the present invention is unique in its ability to exert an effect at the site of guanine modifications. In particular, the water soluble spinach extract of the present invention is able to prevent the conversion of guanine to 8-OHdG, reduce the number of times guanine is converted to 8-OHdG, and/or repair guanine residues that already have been converted to 8-OHdG. This is significant because although spinach is known to have antioxidant properties and previously has been shown to have an ability to repair some types of DNA damage, it is not known that a water soluble spinach extract can be used to prevent or repair damage at guanine residues and/or damage caused by 8-OHdG.

For example, in 1988 Doetsch et al., reported isolation of a novel DNA repair enzyme from spinach. The enzyme was named nuclease SP and it preferentially exerts its effect on adenine mutations. Doetsch et al., “Nuclease SP: a novel enzyme from spinach that incises damaged duplex DNA preferentially at sites of adenine.” Nucleic Acids Res. 1988; 16(14):6935-6952. In addition, Oleykowski et al. has shown that nuclease SP, initially described by Doetsch in 1988, incises at all DNA mismatches except guanine residues. Oleykowski et al., “Incision at nucleotide insertions/deletions and base pair mismatches by the SP nuclease of spinach.” Biochem. 1999; 38(7):2200-5. In contrast, the water soluble spinach extract of the present invention exerts its effect at the site of guanine modifications, as indicated by Example 4, which discusses repair of DNA damage caused by 8-OHdG.

DNA integrity is critical for normal cell operations. The human mitochondrion contains 5-10 identical, circular molecules of DNA. Each consists of 16,569 base pairs carrying the information for 37 genes which encode: 2 different molecules of ribosomal RNA (rRNA); 22 different molecules of transfer RNA (tRNA) (at least one for each amino acid); and 13 polypeptides. The 13 polypeptides are subunits of the protein complexes in the inner mitochondrial membrane, including subunits of NADH dehydrogenase, cytochrome coxidase, and ATP synthase. The damage and incomplete repair of the mtDNA responsible for these critical cofactors and enzymes that occurs during aging can lead to severe loss of energy production, crippling the ability of the cell to produce vital proteins, lipids, and carbohydrates needed for normal functioning of the cell. Thus, there is a need in the art for a composition that can prevent damage of DNA, for example mitochondrial DNA, and/or repair damage of DNA.

The present invention is based on the surprising discovery that a water soluble fraction of spinach, that is, a water soluble spinach extract, possesses the ability to both protect a cell, particularly the DNA in a cell, including nuclear and mitochondrial DNA, from oxidative stress and repair damage caused by ROS, for example, peroxide radicals. Specifically, as described in Example 3, flow cytometry was used to measure the oxidation of superoxide, and thereby demonstrate that the water soluble spinach extract protects DNA (nuclear, mitochondrial, or both) from oxidative stress. As described in Example 4, the same extract is shown to aid in DNA repair. In addition, as discussed in Example 2, the invention is based on the surprising discovery that the water soluble spinach extract is effective at increasing production of ATP, which may be a direct result of protecting the cells from oxidative damage generated during normal electron processes.

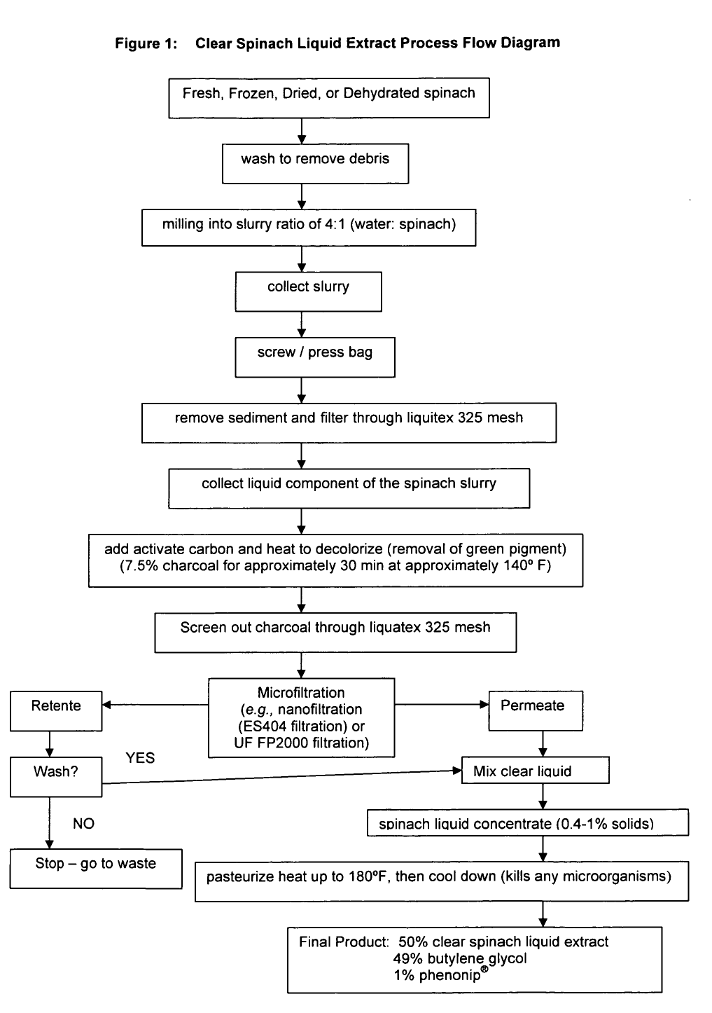

The water soluble spinach extract of the present invention may be obtained, for example, by an extraction method using hot water and a microfiltration step. The microfiltration step might use a variety of different filtration techniques, for example nanofiltration or ultrafiltration. A water soluble spinach extract of the present invention also may be obtained by steam distillation. One of ordinary skill in the art will appreciate that there are numerous other methods that might be used to obtain a water soluble spinach extract that are within the scope of the present invention. One example of an extraction process that may be used to obtain a water soluble spinach extract is described in Example 1 and diagrammed in FIG. 1. Generally this process involves obtaining spinach, fresh, dried, dehydrated, or frozen, and milling the spinach with water into a slurry. The slurry liquids and solids are then separated and the liquid portion of the slurry is collected. The liquid portion of the slurry is added to a steam kettle along with active carbon. Steam is turned on or hot water is added and the liquid portion of the slurry and active carbon are mixed with steam (or hot water) to decolorize the green liquid portion of the slurry to obtain a colorless liquid spinach extract. The liquid spinach extract and active carbon are then separated. Any permeates in the liquid spinach extract may be combined with washed water via microfiltration (for example, nanofiltration or ultrafiltration (“UF”)) for further extraction. The clear liquid spinach extract is then pasteurized in a steam kettle. Preservatives, for example 2-phenoxyethanol such as Phenonip® (Clariant Corp. Charlotte, N.C.) or chlorphenesin such as Germazide® M (Engelhard Corp. Iselin, N.J.), carriers, for example butylene glycol (1,3-Butanediol; 1,3-Butylene glycol), and/or other cosmetic adjuvants, additives, excipients, etc. may be added to the clear liquid spinach extract.

It should be appreciated that certain aspects of the above-identified extraction process may be modified yet still be within the scope of the present invention. For example, the type of steam kettle may be varied, the steam temperatures in the steam kettle may be varied and/or hot water may be used instead of steam, the preservatives used may be varied, the screens or membranes used for filtration may be varied, the amount of water used in preparing the slurry may be varied, the source of the spinach may be varied, the spinach may be dried, dehydrated, fresh, or frozen, etc.

A composition of the present invention comprising the water soluble spinach extract may be administered with an acceptable carrier, additives, preservatives, excipients, and/or cosmetic adjuvants. Further, a composition of the present invention comprising the water soluble spinach extract could be externally administered with an acceptable carrier in the form of a gel, lotion, cream, tonic, emulsion, paste etc. As another example, a composition of the present invention comprising the water soluble spinach extract could be internally administered with an acceptable carrier in the form of a pill, tablet, powder, bar, beverage, etc. Thus, the compositions described herein are useful in a wide variety of finished products, including cosmetic products, pharmaceutical products, food products, and beverage compositions. The finished products in which the compositions of the invention are useful may be used for repairing damage of nuclear DNA, mitochondrial DNA, or both, and/or for preventing damage to DNA, nuclear, mitochondrial, or both, from, for example, ROS or 8-OHdG.

Thus, in one example of the invention, a composition of the present invention is topically administered in the form of a: solution, gel, lotion, cream, ointment, oil-in-water emulsion, water-in-oil emulsion, stick, spray, paste, mousse, tonic, or other cosmetically and topically suitable form.

For example, a composition of the present invention may be administered in a topical formulation and may include a water soluble spinach extract as discussed herein, along with a liposome useful for improving, restoring, and/or maintaining mitochondrial function. Liposomes are known to be efficient drug delivery systems for topical applications in cosmetic and dermatological products. In particular, a liposome is a spherical vesicle with a membrane composed of a phospholipid bilayer. The lipid bilayer of a liposome can fuse with other bilayers, for example cellular and/or mitochondrial membranes, thus delivering the liposome contents.

Liposomes can be composed of a variety of phospholipids including naturally-derived phospholipids with mixed lipid chains such as egg phosphatidylethanolamine, or of pure components like DOPE (dioleolylphosphatidylethanolamine). Liposomes typically are small in size, falling in the range of about 25 to 1000 nm. Liposomes are closed structures composed of a phospholipid bilayer and are capable of encapsulating water-soluble, hydrophilic molecules in their aqueous core and oil-soluble, hydrophobic molecules in the hydrophobic region of the bilayer. Generally, a liposome may be neutral, negative or positive. For example, a positive liposome may be formed from a solution containing phosphatidylcholine, cholesterol, cardiolipin and phosphatidyl serine. Liposomes can be a mixture of multilamellar vesicles and unilamellar vesicles.

As explained above, liposomes are comprised of phospholipids. Phospholipid molecules have a “headgroup” which is hydrophilic in nature and a hydrophobic “tail” consisting of two acyl chains. Aqueous solubility of a phospholipid depends on both the length of the hydrophobic tail and the affinity of the headgroup to water. For example, pure lipids with each acyl chain containing 14 or more carbons in the form of a straight chain (unbranched) with saturated C—C are water insoluble. Generally, as the acyl chain-length of the lipids increases, the critical micelle concentration decreases rapidly.

A liposome used in compositions of the present invention may be primarily comprised of lipids present in a cellular membrane, including phospholipids, ceramides, sphingolipids, cholesterol, and triglycerides, or other lipids such as phytosterols from plants. In one example, a liposome used in compositions of the present invention may be composed primarily of phosphatidylcholine (or phosphatidyl choline). Phosphatidylcholine is a phospholipid that is a major constituent of cell membranes. Phosphatidylcholine is also known as 1,2-dihexadecanoyl-sn-glycero-3-phosphocholine, PtdCho and lecithin. Unsaturated phosphatidylcholine contains choline, omega-6 unsaturated fatty acid (e.g. linoleic acid), omega-3 fatty acids (e.g. gamma-linolenic acid) and has a low level (or absence) of residual glycerides.

Advances in liposome research have enabled liposomes to avoid detection by the body's immune system, specifically, the cells of reticuloendothelial system (RES). Such liposomes are known as “stealth liposomes,” and are constructed with PEG (Polyethylene Glycol) as coating. The PEG coating, which is inert in the body, allows for longer circulatory life for the drug delivery mechanism. Thus, a composition of the present invention may be comprised of a water soluble spinach extract and a liposome primarily composed of phosphatidylcholine and embedded with cardiolipin, for example tetraoleoyl-cardiolipin, and at least one antioxidant, for example methylgentisate and/or I-carnosine, and may additionally comprise a PEG “stealth” coating. In addition to a PEG coating, a stealth liposome used in compositions of the present invention may also have a ligand attached that enables binding to a targeted site of delivery.

In skin care or cosmetic products, liposomes can be formulated in an appropriate matrix (e.g. an acceptable carrier) such as serums, lotions, gels, or creams. More specifically, a composition of the present invention for topical administration might be a cream, lotion, gel, paste, or other cosmetically and topically suitable form and may be comprised of a water soluble spinach extract and a liposome embedded with cardiolipin and antioxidants. In one example, a composition of the present invention for topical administration comprises a water soluble spinach extract, a liposome comprised primarily of phosphatidyl choline and embedded with cardiolipin, for example, tetraoleoylcardiolipin, and at least one antioxidant. The at least one antioxidant may be I-carnosine, methyl gentisate, or both. The cardiolipin embedded in the liposome also might be one or more of tetrapalmitoleoyl-cardiolipin and tetramyristoyl-cardiolipin. The cardiolipin is embedded in the phospholipid bilayer of the liposome. The at least one antioxidant may be embedded in the phospholipid bilayer of the liposome, the aqueous center of the liposome, or both. In one example, at least one antioxidant may be a water soluble antioxidant. In another example, at least one antioxidant may be a lipid soluble antioxidant. In a further example, at least one antioxidant may be a singlet-oxygen scavenger. In a further example, an antioxidant included in the composition of the present invention may be either both water soluble and a singlet-oxygen scavenger or lipid soluble and a singlet-oxygen scavenger.

It is believed that the at least one antioxidant stabilizes the cardiolipin until the cardiolipin is delivered to a cell, for example by topical administration of a composition comprising a liposome containing cardiolipin and at least one antioxidant. In one example, the at least one antioxidant stabilizing the cardiolipin is methylgentisate, a powerful antioxidant that protects the cardiolipin from oxidation. Further, by the nature of liposomal delivery technology, liposomes contain a water interior. To avoid oxidation of cardiolipin from within the liposome, a second antioxidant, for example, I-carnosine, which is a powerful peptide based water soluble antioxidant, may be included in the liposome.

Improved cellular function is accomplished by a composition of the present invention comprising a water soluble spinach extract and a liposome, for example a phosphatidylcholine liposome, comprising cardiolipin, for example tetraoleoyl cardiolipin, and at least one antioxidant because such a composition comprises a water soluble spinach extract to repair and restore nuclear and mitochondrial DNA, an antioxidant, for example, I-carnosine, to protect the cell from oxidative damage, and a cardiolipin, for example, tetraoleoyl-cardiolipin, to improve, maintain or restore mitochondrial function, and/or to repair mitochondrial membranes. Thus, such a composition of the present invention is useful for improving, maintaining or restoring mitochondrial function and for repairing oxidative damage to DNA.

Cardiolipin is a phospholipid of unusual structure and is particularly rich in unsaturated fatty acids. Typically, linoleic acid represents at least 85% of the unsaturated fatty acids present in cardiolipin. Thus, in one example, cardiolipin composed of approximately 85% lineolic acid is embedded in a liposome, for example in the phospholipid bilayer of the liposome. In another example, tetraoleoyl-cardiolipin is embedded in the liposome. Tetraoleoyl-cardiolipin is composed of four oleic acid constituents (C18:1, tetraoleoyl-cardiolipin), which are less susceptible to oxidative damage and break down than lineolic acid cardiolipins. In other examples, the cardiolipin may be seed oil derived cardiolipin. Other examples of cardiolipin that can be used are available, for example, from Avanti® Polar Lipids, Inc. (Alabaster, Ala.). Examples of cardiolipin available from Avanti® Polar Lipids, Inc. include the following: 1,1′,2,2′-Tetramyristoyl Cardiolipin (Ammonium Salt) (Prod. No. 770332); 1,1′,2,2′-Tetramyristoyl Cardiolipin (Sodium Salt) (Prod. No. 750332 or 710335); 1,1′-Oleoyl-2,2′-(12-biotinyl(aminododecanoyl)) Cardiolipin (Ammonium Salt) (Prod. No. 860564); Cardiolipin (E. Coli, Disodium Salt) (Prod. No. 841199); Cardiolipin (Heart, Bovine-Disodium Salt) (Prod. No. 770012); Cardiolipin (Heart, Bovine-Disodium Salt) (Prod. No. 840012); Cardiolipin, Hydrogenated (Heart, Bovine-Disodium Salt) (Prod. No. 830057); Dilysocardiolipin (Heart, Bovine-Disodium Salt) (Prod. No. 850082); Dilysocardiolipin (Heart-Sodium Salt); Heart Cardiolipin Hydrogenated; Lysocardiolipin; Monolysocardiolipin (Heart, Bovine-Disodium Salt) (Prod. No. 850081); and Monolysocardiolipin (Heart-Sodium Salt). In a further example, the cardiolipin embedded in the liposome may be diphosphatidylglycerol or more precisely 1,3-bis(sn-3′-phosphatidyl)-sn-glycerol.

There are many antioxidants that may be incorporated in the compositions of the present invention. For example, an antioxidant embedded in a liposome of the present invention might be water-soluble and thus embedded in the aqueous center of the liposome. In another example, an antioxidant embedded in a liposome might be lipid soluble and thus embedded in the lipid bilayer of the liposome. In a further example, an antioxidant embedded in a liposome of the present invention might be a singlet-oxygen scavenger. In another example, the antioxidant may be both water soluble and a singlet-oxygen scavenger or both lipid soluble and a singlet-oxygen scavenger. In a further example, more than one antioxidant might be embedded in the liposome of the present invention. For example, one or more of the following antioxidants might be embedded in the liposome of the present invention: methylgentisate, I-carnosine, butylated hydroxytoluene (BHT), tert-butylhydroquinone (TBHQ), or some combination thereof.

In one example, methylgentisate is used as an antioxidant because methylgentisate is lipid soluble and has the ability to stabilize cardiolipin. Methylgentisate has a high oxygen radical absorbency capacity (“ORAC”) (25,605 μmol Teq/g), which indicates it is a strong antioxidant. It is possible then to select an antioxidant for use in the present invention based on its ORAC value, for example by selecting antioxidants with high ORAC values. In another example, I-carnosine is used as an antioxidant in the present invention because it is water soluble and has the ability to protect cardiolipin from oxidative damage due to the aqueous center of the liposome. Thus, in one example, the aqueous center of the liposome contains I-carnosine. In a further example, the phospholipid bilayer of the liposome is embedded with cardiolipin and methylgentisate and the aqueous center of the liposome contains I-carnosine.

In another example, an antioxidant embedded in a liposome of the present invention might be an antioxidant found in mitochondria, such as for example, glutathione. It has been shown that when glutathione is artificially depleted from cells, oxidative damage increases. The level of glutathione in mitochondria might be even more important than the level of glutathione in the rest of the cell. Mitochondrial glutathione levels diminish more with age than do the levels in the rest of the cell. This decline seems to make mitochondria more susceptible to oxidative damage.

Ascorbic acid (i.e. vitamin C) and vitamin E (i.e. tocopherol) are other examples of antioxidants that might be embedded in a liposome that is administered in a composition of the present invention containing both the liposome and a water soluble spinach extract. It should be appreciated that there are numerous other antioxidants that might be embedded in a liposome that is administered in a composition of the present invention. It also should be appreciated that the liposome used in the present invention, that is a liposome comprising a cardiolipin and at least one antioxidant, for example methylgentisate, I-carnosine, or both, has an advantage of being stable and not susceptible to substantial oxidative damage when stored at temperatures ranging from approximately 10° C. to approximately 60° C., desirably from approximately 20° C. to approximately 55° C., desirably from approximately 30° C. to approximately 50° C.

Compositions of the present invention that are suitable for topical administration may be mixed with an acceptable carrier. An acceptable carrier may act variously as solvent, carrier, diluent or dispersant for the constituents of the composition, and allows for the uniform application of the constituents to the surface of the skin at an appropriate concentration. The acceptable carrier may also facilitate penetration of the composition into the skin.

In one example of a formulation for topical application that includes a water soluble spinach extract of the present invention, the acceptable carrier may form from about 80% to about 100% by weight of the total composition. In other examples, the acceptable carrier may form from about 85% to about 95% by weight of the total composition. Thus, in one example a composition of the present invention may comprise from approximately 0.01% to approximately 5% water soluble spinach extract, from approximately 0.01% to approximately 5% liposome containing cardiolipin and at least one antioxidant; and approximately 90% to approximately 99.99% carrier. In another example, a composition of the present invention may comprise from approximately 0.01% to approximately 5% by weight of the total composition water soluble spinach extract and approximately 90% to approximately 99.99% by weight of the total composition acceptable carrier. In another example, a composition of the present invention may comprise 90% to approximately 99.99% by weight of the total composition acceptable carrier, for example butylene glycol, including any additives and/or excipients, for example a preservative, such as 2-phenoxyethanol or Phenonip®, or chlorphenesin or Germazide® M, forming from approximately 0.01% to approximately 5% by weight of the total composition, and approximately 0.01% to approximately 5% by weight of the total composition water soluble spinach extract. One of ordinary skill in the art will appreciate that when a preservative is included in a composition of the present invention, the type of preservative included will determine the concentration of preservative. For example, Phenonip® is a commercially available preservative that will preserve cosmetics and toiletries when incorporated at concentrations ranging from approximately 0.25% to approximately 1% by weight of the total composition. Another commercially available preservative that might be used is Germazide® M, which is described more fully in U.S. Pat. No. 6,447,793. Germazide® M typically is useful at ranges of approximately 0.5% to approximately 2% by weight of the total composition.

In other examples or formulations for topical application of a composition of the present invention that includes at least a water soluble spinach extract and that may also include a liposome embedded with cardiolipin and at least one antioxidant, the acceptable carrier forms from about 90% to about 99.99% by weight of the total composition; from about 97% to 99% by weight of the total composition; from about 91% to about 98% by weight of the total composition; from about 92% to about 97% by weight of the total composition; from about 93% to about 96% by weight of the total composition; or from about 94% to about 95% by weight of the total composition. The acceptable carrier can, in the absence of other cosmetic adjuncts or additives, form the balance of the composition.

The water soluble spinach extract and other ingredients used in practicing the present invention may be soluble or insoluble in the acceptable carrier. If all ingredients of a composition are soluble in the acceptable carrier, then the carrier acts as solvent. However, if all or some ingredients of a composition are insoluble in the acceptable carrier, then those ingredients are dispersed in the carrier by means of, for example, a suspension, emulsion, gel, cream or paste, and the like.

Thus, it will be apparent to the skilled artisan that the range of possible acceptable carriers is very broad. For example, acceptable carriers can be emulsions, lotions, creams, or tonics. Acceptable carriers can comprise water, ethanol, butylene glycol, or other various solvents that aid in penetration of the skin. Some examples of suitable carriers are described in U.S. Pat. No. 6,184,247 and in U.S. Pat. No. 6,579,516, the entire contents of which are incorporated herein by reference.

In general, acceptable carriers according to the present invention may comprise, but are not limited to comprising, any of the following examples: water; butylene glycol; castor oil; ethylene glycol monobutyl ether; diethylene glycol monoethyl ether; corn oil; dimethyl sulfoxide; ethylene glycol; isopropanol; soybean oil; glycerin; soluble collagen; zinc oxide; titanium oxide; or Kaolin.

In one aspect, the acceptable carrier used in practicing the present invention comprises water and ethanol. Optionally, the acceptable carrier also contains butylene glycol and/or frescolate MGA. For example, the acceptable carrier can comprise 40-60% water, 45-55% ethanol, and 5-10% butylene glycol by weight of the composition. In practicing the present invention, the acceptable carrier is mixed with the water soluble spinach extract comprising from approximately 0.01% to approximately 5% by weight of the total composition; more specifically from approximately 1% to approximately 5% by weight of the total composition; more specifically from approximately 2% to approximately 4% by weight of the total composition; more specifically approximately 3% by weight of the total composition.

Additionally, acceptable carriers used in the present invention may optionally comprise one or more humectants, including but not limited to: dibutyl phthalate; soluble collagen; sorbitol; or sodium 2-pyrrolidone-5-carboxylate. Other examples of humectants that may be used in practicing the present invention can be found in the CTFA (Cosmetic Toiletry and Fragrance Association) Cosmetic Ingredient Handbook, the relevant portions of which are incorporated herein by reference.

Additionally, acceptable carriers in the present invention may optionally comprise one or more emollients including but not limited to: butane-1,3-diol; cetyl palmitate; dimethylpolysiloxane; glyceryl monoricinoleate; glyceryl monostearate; isobutyl palmitate; isocetyl stearate; isopropyl palmitate; isopropyl stearate; butyl stearate; isopropyl laurate; hexyl laurate; decyl oleate; isopropyl myristate; lauryl lactate; octadecan-2-ol; caprylic triglyceride; capric triglyceride; polyethylene glycol; propane-1,2-diol; triethylene glycol; sesame oil; coconut oil; safflower oil; isoamyl laurate; nonoxynol-9; panthenol; hydrogenated vegetable oil; tocopheryl acetate; tocopheryl linoleate; propylene glycols; arachis oil; castor oil; isostearic acid; palmitic acid; isopropyl linoleate; lauryl lactate; myristyl lactate; decyl oleate; or myristyl myristate. Other examples of emollients that may be used in practicing the present invention can be found in the CTFA Cosmetic Ingredient Handbook, the relevant portions of which are incorporated herein by reference.

Additionally, acceptable carriers used in the present invention may optionally comprise one or more penetration enhancers including but not limited to: pyrrolidones, for example 2-pyrrolidone; alcohols, such as ethanol; alkanols, such as decanol; glycols, such as propylene glycol, dipropylene glycol, butylene glycol; surfactants; or terpenes.

Other acceptable carriers that may be used in practicing the present invention will be apparent to those of skill in the art and are included within the scope of the present invention.

In another example, a composition of the present invention is administered orally in the form of a liquid or a solid. The liquid may be water-based, milk-based, tea-based, fruit juice-based, or some combination thereof. Solid and liquid compositions for internal administration according to the present invention can further comprise thickeners, including xanthum gum, carboxymethyl-cellulose, carboxyethylcellulose, hydroxypropylcellulose, methylcellulose, hydroxypropylmethylcellulose, microcrystalline cellulose, starches, dextrins, fermented whey, tofu, maltodextrins, polyols, including sugar alcohols (e.g., sorbitol and mannitol), carbohydrates (e.g. lactose), propylene glycol alginate, gellan gum, guar, pectin, tragacanth gum, gum acacia, locust bean gum, gum arabic, gelatin, as well as mixtures of these thickeners. These thickeners are typically included in the compositions of the present invention at levels up to about 0.1%, depending on the particular thickener involved and the viscosity effects desired.

The solid and liquid (food, beverage, supplement or pharmaceutical) compositions of the present invention can, and typically will, contain an effective amount of one or more sweeteners, including carbohydrate sweeteners and natural and/or artificial no/low calorie sweeteners. The amount of the sweetener used in the compositions of the present invention will vary, but typically depends on the type of sweetener used and the sweetness intensity desired.

The compositions of the present invention, regardless of the mode of administration, may also contain various known and conventional cosmetic adjuvants so long as they do not detrimentally affect the desired repair of damage to nuclear DNA, mitochondrial DNA, or both, and/or prevention of damage to such DNA from, for example, reactive oxygen species or 8-OHdG. For example, a composition of the present invention can further include one or more additives or other optional ingredients well known in the art, which can include but are not limited to fillers (e.g., solid, semi-solid, liquid, etc.); carriers; diluents; thickening agents; gelling agents; vitamins, retinoids, and retinols (e.g., vitamin B₃, vitamin A, etc.); pigments; fragrances; sunscreens and sunblocks; antioxidants and radical scavengers; organic hydroxy acids; exfoliants; skin conditioners; moisturizers; ceramides, pseudoceramides, phospholipids, sphingolipids, cholesterol, glucosamine, pharmaceutically acceptable penetrating agents (e.g., n-decylmethyl sulfoxide, lecithin organogels, tyrosine, lysine, etc.); antimicrobial agents; amino acids such as proline, pyrrolidone carboxylic acid, its derivatives and salts, saccharide isomerate, panthenol, buffers together with a base such as triethanolamine or sodium hydroxide; waxes, such as beeswax, ozokerite wax, paraffin wax; plant extracts, including but not limited to Aloe Vera, cornflower, witch hazel, elderflower, or cucumber; opacifiers; suspending agents; binders; preservatives; and combinations thereof. One example of a preservative that might be included is Phenonip® (Clariant, Charlotte, N.C.), an anti-microbial mixture of plant extracts including 2-phenoxyethanol and glycol ethers. Other suitable additives and/or adjuncts are described in U.S. Pat. No. 6,184,247, the entire contents of which are incorporated herein by reference.

The composition can include additional inactive ingredients, including, but not limited to surfactants, co-solvents, and excipients. Surfactants, such as hydrophilic and hydrophobic surfactants, can be included in the compositions. Particular surfactants can be used based on the on the overall composition and the intended delivery of the composition. Useful surfactants include polyethoxylated (PEG) fatty acids, PEG-fatty acid diesters, PEG-fatty acid mono- and di-ester mixtures, polyethylene glycol glycerol fatty acid esters, alcohol-oil transesterification products, polyglycerized fatty acids, propylene glycol fatty acid esters, mixtures of propylene glycol esters-glycerol esters, mono- and diglycerides, sterol and sterol derivatives, polyethylene glycol sorbitan fatty acid esters, polyethylene glycol alkyl ethers, polysaccharide esters, polyethylene glycol alkyl phenols, polyoxyethylene-polyoxypropylene block copolymers, sorbitan fatty acid esters, lower alcohol fatty acid esters, ionic surfactants, and mixtures thereof.

Other additives that may be included in compositions of the present invention will be apparent to those of skill in the art and are included within the scope of the present invention.

Regardless of the mode of administration, generally, the compositions of the present invention may be administered at least on a daily basis. Administration of the compositions of the invention may continue for any suitable period of time. It should be appreciated that the degree of repair of damage to nuclear DNA, mitochondrial DNA, or both, and/or degree of prevention of damage to such DNA from, for example, reactive oxygen species or 8-OHdG, will vary directly with the total amount and frequency of composition used.

In one example, a composition of the present invention is administered at least once a day. In another example, a composition of the present invention may be administered twice daily. In a further example, a composition of the present invention may be administered three to five times daily. In another example, there is no limit on the amount of a composition of the present invention that might be administered daily. For best effect, compositions of the present invention are administered on at least a daily basis for at least a week to several weeks. Compositions of the present invention also may be administered on at least a daily basis for several weeks to a month to several months to a year to years. It should be appreciated that there is no limit on how frequently or how long the composition of the present invention is administered.

It is intended that the foregoing detailed description be regarded as illustrative rather than limiting. The present invention is further illustrated by the following experimental investigations and examples, which should not be construed as limiting. The contents of all references, patents and published applications cited throughout this patent are hereby incorporated by reference herein.

### EXAMPLES

### Example 1

### Process for Extracting Spinach to Obtain Water Soluble Spinach Extract

Spinach, grown, for example, under organic conditions, is harvested and the spinach leaves are washed under gentle conditions. Washed leaves are then air dried. When dried, the spinach is milled with water into a slurry using a Rietz Disintegrator (Hosokawa) with ½ openings; at a ratio of 4:1, water to fresh spinach.

The liquids and solids are then separated using suitable equipment such as a bag press and Liquatex unit (with USSS 325 Mesh sieve, approximately 44 micron opening).

The spinach extract liquid and 7.5% of active carbon are added to a steam kettle. Heat, in the form of hot water or steam at approximately 140° F. and is used to mix the spinach extract liquid and 7.5% of active carbon for approximately 30 minutes. An agitator also may be used for mixing. This heating step should decolorize the green spinach extract liquid, which may be useful when adding the water soluble extract to topical cosmetics.

The extract liquid and activate carbon are separated, employing suitable equipment such as a Liquatex unit (with USSS 325 Mesh sieve, approximately 44 micron opening). Additional liquid can be separated and clarified from the activate carbon by employing suitable UF filtration equipment, such as membrane # FP200.

The permeates are combined with washed water via UF filtration. Total solids in the resulting liquid concentrate are in the 0.4-1.0% range. The clear spinach liquid concentrate is pasteurized in a steam kettle at 180° F. with agitator. Preservatives, for example 2-phenoxyethanol (e.g. Phenonip® (Clariant Corp. Charlotte, N.C.)) or chlorphenesin (e.g. Germazide® M (Engelhard Corp. Iselin, N.J.), are added to the clear spinach liquid concentrate. The final formula is % wt/wt/: approximately 50% clear spinach liquid concentrate; approximately 49% carrier (butylene glycol) and approximately 1% preservative (for example, Phenonipe (2-phenoxyethanol) or Germazide® M (chlorphenesin)).

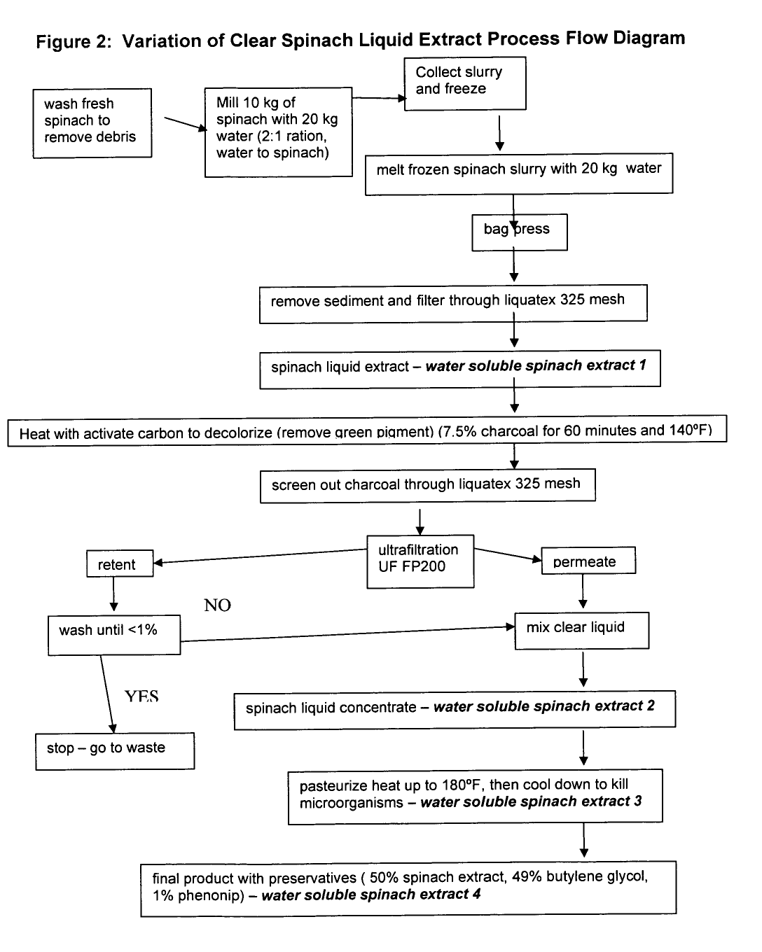

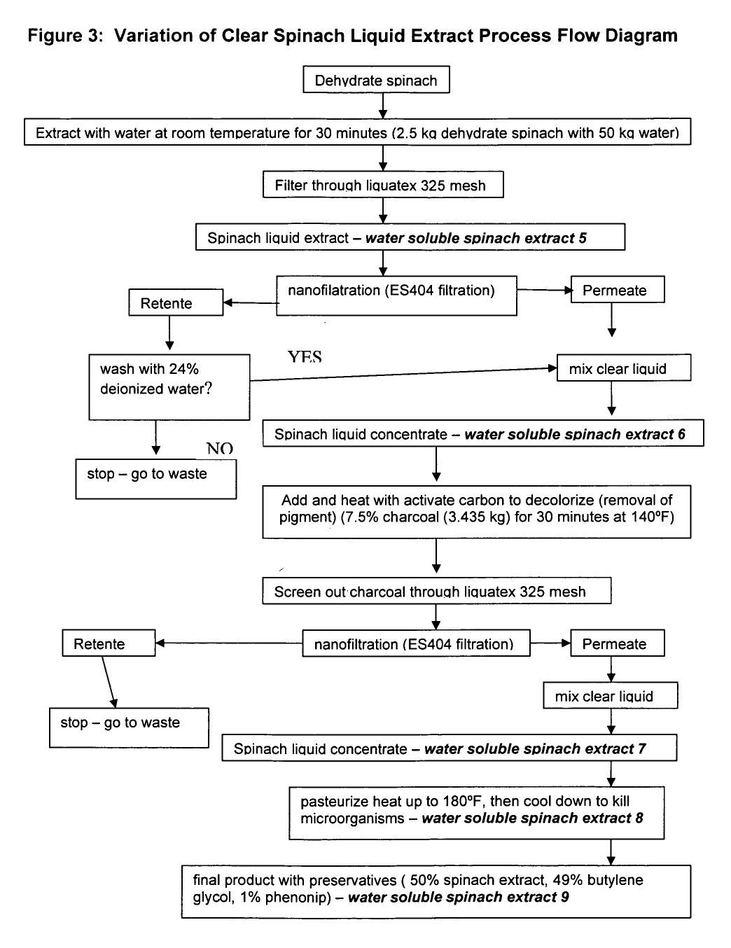

FIG. 2 illustrates a variation of the above-described process that may be used to obtain a water soluble spinach extract using frozen spinach as a starting material. FIG. 2 also illustrates at what point in the extraction process water soluble extracts 1-4, referenced below in Table II were collected. FIG. 3 also illustrates a variation of the above-described process that may be used to obtain a water soluble spinach extract using dehydrate spinach as a starting material. FIG. 3 also illustrates at what point in the extraction process water soluble extracts 5-9, referenced below in Table II were collected.

### Example 2

### Measurement of ATP Levels

The level of cellular ATP is a marker of cellular and mitochondrial health. As explained in this example, ATP levels can be monitored using an ATP dependent luciferase that generates light in the presence of ATP. The amount of light generated is directly proportional to the amount of ATP present.

CHO-K1 (Chinese hamster ovary) cells are purchased from ATCC (Manassas, Va.) (cell accession # ATCC CCL 61). Cell cultures are established in 96 well opaque plates with 1×10⁴ cells per well. Following adherence, the cells are fed low glucose media (1 g/l) and are incubated overnight. Cells cultured in low glucose media have a reduced ATP content.

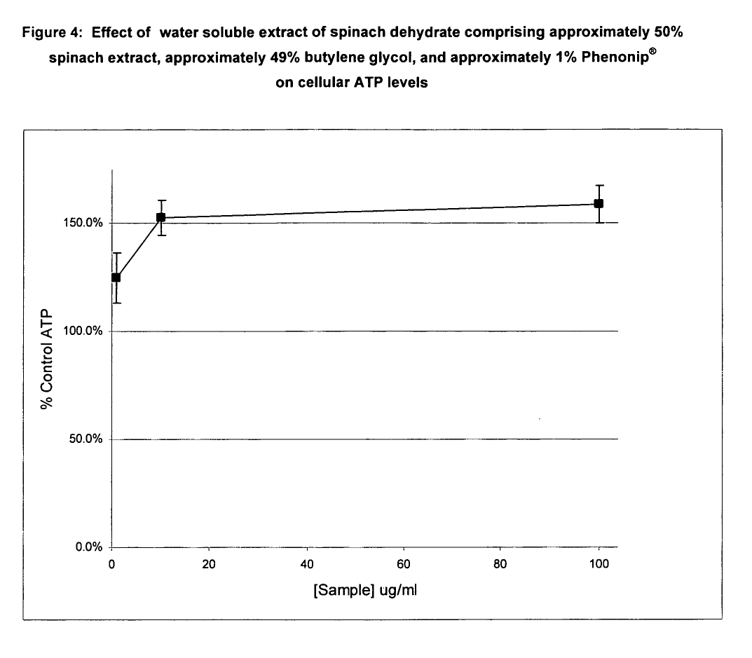

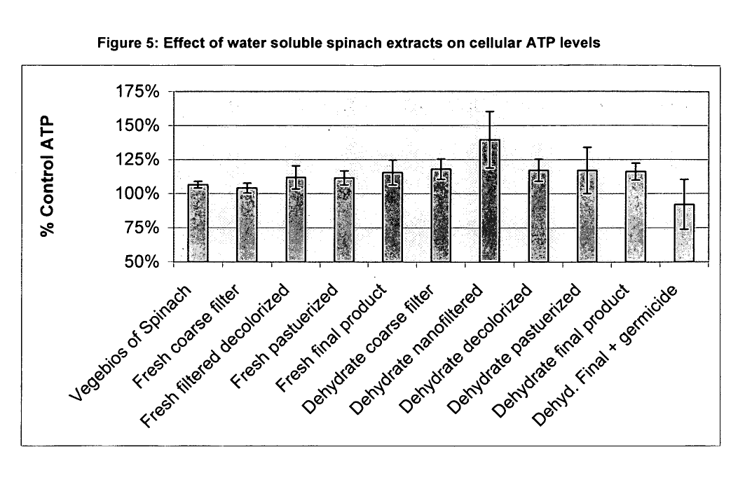

Following overnight culture, the cells are exposed to t-butyl-peroxide (1 mM) for 2 hours to induce cellular stress. Peroxide reduces ATP levels lower than glucose alone and mimics a damaged cell state. Following peroxide exposure, fresh low glucose media is added back to the cells. Immediately following challenge with peroxide, the cells are exposed to test samples, typically at 1, 10, and 100 mg/ml final concentration. The cells are incubated with the test samples for 4 hours at 37° C. The test samples used in this example include samples derived from a fresh frozen spinach slurry and a spinach dehydrate. The samples from the extraction process for both types of spinach starting material include coarse filtered spinach extract, nano-filtered extract, decolorized extracted, pasteurized extract, and final product. The results are reported at FIGS. 4 and 5. In FIGS. 4 and 5, data are expressed as % control ATP compared to the ATP levels in untreated control cells. Therefore, an increase in ATP is considered a positive effect of the sample(s). The data presented in FIG. 5 shows that samples derived from the spinach dehydrate tend to have more activity than those derived from fresh frozen spinach.

This assay screens compounds for the ability to restore ATP levels following stressed conditions mimicking a damaged cell state. Therefore, following incubation of the challenged cells with the test samples, the relative cellular ATP levels are measured using the Cell-Titer Glo reagent from Promega (Madison, Wis.) according to the manufacturer's specifications. Briefly, the cells are equilibrated to room temperature at which time the media is flicked out of the wells. The diluted reagent is added to the wells and the plate is incubated at room temperature for 15 minutes. Luminescence is read on a Wallach plate reader.

The mean luminescence of each treatment group is calculated and % untreated control is determined by dividing the mean from each test group by the mean of the untreated control. The untreated control is considered 100% so any result above 100% is considered a net positive increase in cellular ATP levels. The results of this example are reported at FIGS. 4 and 5.

### Example 3

### Measurement of Protection from Oxidative Stress

The assay described in this experiment measures oxidative stress occurring within the mitochondria. As discussed above, the biochemical reactions used by mitochondria to generate energy yielding ATP molecules also produce highly oxidizing superoxide free radical as a by-product. Using flow cytometry, the below example measures the protection various test samples provide from oxidative stress by monitoring the status of superoxide within mitochondria following treatment with the test materials.

MitoSOX Red mitochondrial superoxide indicator (Invitrogen cat#M36008) is a fluorescent dye that is selectively taken up by mitochondria. Once in the mitochondria, MitoSOX reacts with superoxide free radical to form a fluorescent product (excitation/emission maxima=510/580 nm) that binds to mitochondrial nucleic acids. A higher fluorescence reading corresponds to a higher level of superoxide free radical being present within the mitochondria.

Cell Culture: One 6 well plate is used per sample. Multiple plates may be prepared simultaneously. Refer to Table I for a plate description. THP-1 monocytes (ATCC cat# TIB-202) are plated at 1.5×10⁶ cells/well in 6 well plates (contained in 2 ml/well RMPI 1640 media supplemented with 10% FBS) and incubated for 1 hour before treating.

TABLE I
Plate Description

| Well | Description | Contents |
|---|---|---|
| 1 | Positive Control | Load: 2 ml of media with 1.5 × 10⁶ cells |
| | | Treatment: 1 ml of 300 uM GMEE |
| | | Challenge: 1 ml of 5 mM H₂O₂ |
| 2 | Negative Control | Load: 2 ml of media with 1.5 × 10⁶ cells |
| | | Treatment: 1 ml of media |
| | | Challenge: 1 ml of 5 mM H₂O₂ |
| 3 | Normal Control | Load: 2 ml of media with 1.5 × 10⁶ cells |
| | | Treatment: 1 ml of media |
| | | Challenge: 1 ml of media |
| 4 | Sample Replicate | Load: 2 ml of media with 1.5 × 10⁶ cells |
| | #1 | Treatment: 1 ml of 300 ug/ml sample solution |
| | | Challenge: 1 ml of 5 mM H₂O₂ |
| 5 | Sample Replicate | Load: 2 ml of media with 1.5 × 10⁶ cells |
| | #2 | Treatment: 1 ml of 300 ug/ml sample solution |
| | | Challenge: 1 ml of 5 mM H₂O₂ |
| 6 | Sample Replicate | Load: 2 ml of media with 1.5 × 10⁶ cells |
| | #3 | Treatment: 1 ml of 300 ug/ml sample solution |
| | | Challenge: 1 ml of 5 mM H₂O₂ |

Treatment: Glutathione monoethylester (GMEE, 100 uM final concentration) is used as a positive control. More specifically, the positive control is prepared by making a 1.2 mM stock solution by dissolving 4 mg glutathione monoethylester in 10 ml media (RMPI 1640 media supplemented with 10% FBS). The positive control working solution (300 uM) is then prepared by adding 2.5 ml of the stock solution to 7.5 ml media. One ml of the positive control working solution is added to 1 well of the plate, giving a final treatment concentration of 100 uM glutathione monoethyl ester.

A negative control and a normal control are prepared by adding 1 ml of media (RMPI 1640 media supplemented with 10% FBS) to designated wells on the plate (1 well per control).

The test sample may be prepared by making a sample stock solution at a concentration of 10 mg/ml by weighing 100 mg test sample into a 15 ml-disposable centrifuge tube and adding 10 ml deionized H₂O. In this example, the test sample was a water soluble extract of spinach dehydrate comprising approximately 50% spinach extract, approximately 49% butylene glycol, and approximately 1% preservative, for example Phenonip® or Germazide® M. The test sample stock solution is then serially diluted to give a sample working solution of 300 μg/ml. One ml of the test sample working solution is then added to each of three remaining wells on the plate (triplicate wells), giving a final test sample concentration of 100 ug/ml for each sample well.

The plate containing both test sample and control wells is then incubated 3 hours at 37° C., 5% CO₂.

Challenge: After incubation for 3 hours, the cells in wells containing the test sample, positive control, and negative control are subjected to oxidative stress by introducing a challenge solution The challenge solution is prepared as follows:

852 μl 8.8 M H₂O₂+4.15 ml Media (=1.5 M H₂O₂)

1 ml 1.5 M H₂O₂+9 ml Media (=150 mM H₂O₂)

1 ml 150 mM+30 ml Media (=5 mM H₂O₂)

1 ml 5 mM H₂O₂ is added to all wells of the plate except for the normal control. One ml of media (RMPI 1640 media supplemented with 10% FBS) is added to the normal control well. The plate is then incubated 3 hours at 37° C., 5% CO₂.

Staining: Following incubation with the challenge, the contents of all wells are transferred to flow cytometry tubes and centrifuged at 100×g for 5 minutes. Media above the cells is removed and cells are then stained by adding 1 ml MitoSOX (5 μM) to each tube. The MitoSOX stain is obtained by dissolving 2 vials of MitoSOX (50 μg per vial, Invitrogen cat # M36008) using 13 μL DMSO per vial to give a solution of 5 mM per vial (mw=760 g/mole). The contents of both vials are transferred to 26 ml media (5 μM). The cells are incubated with the stain for 15 minutes at room temperature in the dark.

Flow Cytometry: Following staining, the samples are analyzed by flow cytometry (e.g. Becton-Dickinson FACS Caliber), using 488 nm excitation laser and FL2 588 nm emission filter. The data are linearized to a curve defined by the responses of the negative and normal controls (0% protection=untreated/challenged negative control, 100% protection=untreated/unchallenged normal control). Under these conditions, the positive control (100 uM glutathione methyl ester) provides 35% protection from oxidative stress.

Results: The results of this experiment demonstrate that the water extract of spinach dehydrate provided significant protection, that is an average of 91.3±0.9% protection, from oxidative stress.

### Example 4

### Measurement of 8-OHdG Repair

In this example, cells are challenged with peroxide and UV light to increase levels of 8-OHdG, a marker of oxidative DNA damage. The ability of test samples to aid cells in recovering from the challenge, the length of time to recovery, and repair of the DNA damage are measured.

Specifically, human dermal derived HS27 fibroblasts and HEK001 keratinocytes are purchased from the American Tissue Culture Collection (ATCC) (Manassas, Va.). Co-cultures of HS27 and HEK001 are established in 12 well plates with 4×10⁴ and 8×10⁴/well. The cells are cultured overnight. Following overnight culture, the culture media is replaced with phosphate buffered saline containing 0.5% fetal bovine serum. The cell cultures are then exposed to full spectrum UV light from a solar simulator (at 25 mJ/cm² as measured with a UVB meter) and to H₂O₂ (5 mM) simultaneously to generate the 8-OHdG damage. Following exposure to UV light and H₂O₂, the cells are gently washed and fresh complete media is added. The cells then are treated with the test samples, including nine liquid water soluble extracts of spinach (obtained using the process outlined in FIG. 1), Vegebios of Spinach (an aqueous extract of Spinach leaves using a special method of steam distillation, available from CEP Solabia Group (France) and suppliers such as Aston Chemicals, Aylesbury, UK), and positive control at final concentrations of 1, 10, and 100 μg/ml from a stock solution of 50 mg/ml. Following treatment, the cells are returned to the 37° C. incubator and incubated for 1-2 hours.

Following the incubation period, the cells are washed and collected by trypsin digestion (using TrypI E cell dissociation reagent) and centrifugation. DNA is isolated and collected from the cells using Sigma's GenElute mammalian genomic DNA miniprep kit. Following DNA isolation and collection, the amount of DNA in each sample may be determined using a PicoGreen® dsDNA kit from Molecular Probes (Invitrogen).

An equivalent amount of DNA is digested using nuclease P1 and alkaline phosphatase as recommended by the Japanese Institute for the Control of Aging. The digested samples are assayed using an ELISA kit. Specifically, 8-OHdG is measured in the DNA samples by adding 2 mg DNA from each sample to an 8-OHdG ELISA kit from the Japanese Institute for the Control of Aging (Nikken SEIL Corp., Haruoka, Fukuroi, Shizuoka, Japan).

The results of this example are reported below in Table II. The results are reported as the percentage of 8-OHdG present in cells exposed to the test sample compared to the percentage present in control cells. Therefore, any values lower than 100% are considered positive results and are indicative of repair of DNA damage. Vegebios of Spinach, an aqueous extract of spinach leaves using a special method of steam distillation is available from CEP Solabia Group (France) (or a distributor of Solabia, for example Aston Chemicals (Aylesbury, UK)), and demonstrated a significant ability to repair DNA damage with a percent control value of 63.9%. Water soluble spinach extracts 1, 6, 7, and 8 also demonstrated significant ability to repair DNA damage, with percent control values of 85.7%, 82.1%, 82.0%, and 80.9%, respectively.

TABLE II
8-OHdG DNA Repair

| Test Ingredient: | % Control |
|---|---|
| Commercially available spinach steam distillate (Vegebios of | 63.9% |
| Spinach) | |
| Water Soluble Spinach Extract 1 (fresh coarse filter - FIG. 2) | 85.7% |
| Water Soluble Spinach Extract 2 (fresh filtered decolorized - | 103.1% |
| FIG. 2) | |
| Water Soluble Spinach Extract 3 (fresh pasteurized - FIG. 2) | 131.3% |
| Water Soluble Spinach Extract 4 (fresh final product - FIG. 2) | 118.5% |
| Water Soluble Spinach Extract 5 (dehydrate coarse filter - | 104.9% |
| FIG. 3) | |
| Water Soluble Spinach Extract 6 (dehydrate 1^{st} nanofiltered - | 82.1% |
| FIG. 3) | |
| Water Soluble Spinach Extract 7 (dehydrate decolorized 2^{nd} | 82.0% |
| nanofiltered - FIG. 3) | |
| Water Soluble Spinach Extract 8 (dehydrate pasteurized - | 80.9% |
| FIG. 3) | |
| Water Soluble Spinach Extract 9 (water soluble extract of | 108.9% |
| spinach dehydrate comprising approximately 50% | |
| spinach extract, approximately 49% butylene glycol, | |
| and approximately 1% Phenonip ® - FIG. 3) | |
| Water Soluble Spinach Extract 10 (water soluble extract of | 102.9% |
| spinach dehydrate comprising approximately 50% spinach | |
| extract, approximately 49% butylene glycol, and | |
| approximately 1% Germazide ® M) | |
| Positive Control (with UV) | 100.0% |

The above descriptions are those of the preferred embodiments of the invention. Various alterations and changes can be made without departing from the spirit and broader aspects of the invention as defined in the appended claims, which are to be interpreted in accordance with the principles of patent law including the doctrine of equivalents. Any references to claim elements in the singular, for example, using the articles “a,” “an,” “the,” or “said,” is not to be construed as limiting the element to the singular.

1. A method of repairing oxidative damage to DNA comprising administering a composition comprising a water soluble spinach extract and an acceptable carrier.

2. The method of claim 1, wherein the oxidative damage converts at least one guanine residue in the DNA to 8-hydroxydeoxyguanosine.

3. The method of claim 2, wherein the 8-hydroxydeoxyguanosine causes a base pairing modification in at least one DNA base pair.

4. The method of claim 3, wherein the DNA is nuclear DNA, mitochondrial DNA, or both.

5. The method of claim 1, wherein the water soluble spinach extract is obtained by the process comprising:
forming an aqueous slurry of spinach,
separating a liquid component of the slurry from a solid component of the slurry,
mixing the liquid component of the slurry with active carbon using hot water or steam,
separating the liquid component from the active charcoal, and
microfiltering the liquid component of the slurry to collect a substantially colorless, water soluble extract of spinach.

6. The method of claim 6, further comprising pasteurizing the substantially colorless, water soluble extract of spinach.

7. The method of claim 6, wherein the process further comprises adding at least one preservative to the substantially colorless, water soluble extract of spinach.

8. The method of claim 1, wherein the composition comprising a water soluble spinach extract is topically administered or orally administered.

9. A method of preventing or decreasing oxidative damage of DNA comprising administering a composition comprising a water soluble spinach extract.

10. The method of claim 11, wherein the DNA is nuclear DNA, mitochondrial DNA, or both.

11. The method of claim 9, wherein the water soluble spinach extract is obtained by the process comprising:
forming an aqueous slurry of spinach,
separating a liquid component of the slurry from a solid component of the slurry,
mixing the liquid component of the slurry with active carbon using hot water or steam,
separating the liquid component from the active charcoal, and
microfiltering the liquid component of the slurry to collect a substantially colorless, water soluble extract of spinach.

12. The method of claim 11, further comprising pasteurizing the substantially colorless, water soluble extract of spinach.

13. The method of claim 12, wherein the process further comprises adding at least one preservative to the substantially colorless, water soluble extract of spinach.

14. The method of claim 11, wherein the composition comprising a water soluble spinach extract is topically administered or orally administered.

15. A method of increasing ATP production or synthesis in a cell comprising administering a composition comprising a water soluble spinach extract.

16. The method of claim 15, wherein the water soluble spinach extract is obtained by a process comprising:
forming an aqueous slurry of spinach,
separating a liquid component of the slurry from a solid component of the slurry,
mixing the liquid component of the slurry with active carbon using hot water or steam,
separating the liquid component from the active charcoal, and
microfiltering the liquid component of the slurry to collect a substantially colorless, water soluble extract of spinach.

17. The method of claim 20, further comprising pasteurizing the substantially colorless, water soluble extract of spinach.

18. The method of claim 17, wherein the method of administering the composition comprising a water soluble spinach extract is selected from the group consisting of topical administration, oral administration, and parenteral administration.

19. A composition for improving, maintaining or restoring mitochondrial function and for repairing oxidative damage to DNA comprising a water soluble spinach extract, a liposome, a cardiolipin, and at least one antioxidant, wherein the cardiolipin is embedded in a phospholipid bilayer of the liposome and the at least one antioxidant is embedded in a phospholipid bilayer of the liposome, contained in an aqueous center of the liposome, or both.

20. The composition of claim 19, wherein the liposome is primarily comprised of phosphatidylcholine.

21. The composition of claim 20, wherein the cardiolipin is tetraoleoyl-cardiolipin.

22. The composition of claim 21, wherein the at least one antioxidant is methylgentisate, and further wherein the methylgentisate is embedded in a phospholipid bilayer of the liposome along with the cardiolipin.

23. The composition of claim 22, further comprising a second antioxidant, wherein the second antioxidant is I-carnosine, and further wherein the I-carnosine is contained in an aqueous center of the liposome.

24. The composition of claim 23, further comprising an extract of mustard (Brassica) seed.

25. The composition, of claim 23, further comprising an extract of Arabidopsis thaliana.

26. The composition of claim 25, wherein the extract of Arabidopsis thaliana is 8-oxoguanine DNA glycosylase.
